(19) United States
(12) Patent Application Publication
(10) Pub. No.: US 2024/0173078 A1
(43) Pub. Date: May 30, 2024

(54) DATA PROCESSING METHOD

(71) Applicant: MEDIT CORP., Seoul (KR)

(72) Inventors: Jin Young KIM, Seoul (KR); Sung Hoon LEE, Seoul (KR)

(73) Assignee: MEDIT CORP., Seoul (KR)

(21) Appl. No.: 18/282,086

(22) PCT Filed: Mar. 11, 2022
(86) PCT No.: PCT/KR2022/003441

§ 371 Date: Sep. 14, 2023

(30) Foreign Application Priority Data
Mar. 16, 2021 (KR) 10-2021-0033859
Mar. 11, 2022 (KR) 10-2022-0030565

(51) Int. Cl. A61B 34/10; A61C 7/00; G06T 7/11; G06T 17/00; G06T 19/20

(52) U.S. Cl. CPC A61B 34/10; A61C 7/002; G06T 7/11; G06T 17/00; G06T 19/20

(57) ABSTRACT

A data processing method according to the present disclosure comprises: determining whether a simulation model is to be generated, based on scan data of an object obtained by scanning the object, and at least one simulation condition for the scan data; when it has been determined that the simulation model is not generated, editing at least a part of the scan data; and generating the simulation model based on the edited scan data and the at least one simulation condition previously applied in the determining whether the simulation model is to be generated.

### CROSS REFERENCE TO RELATED APPLICATIONS

This application is a National Stage of International Application No. PCT/KR2022/003441 filed Mar. 11, 2022, claiming priority based on Korean Patent Application No. 10-2021-0033859 filed Mar. 16, 2021 and Korean Patent Application No. 10-2022-0030565 filed Mar. 11, 2022.

### TECHNICAL FIELD

The present disclosure relates to a data processing method, and more particularly, to a data processing method of editing scan data to generate a simulation model.

### BACKGROUND

3D scanning technology is widely used in various industries such as measurement, inspection, reverse engineering, content creation, CAD/CAM for dental treatments, and medical devices, and the practicality thereof has increased due to the improvement of scanning performance due to the development of computing technology. In particular, in the field of dental treatments, since 3D scanning technology is performed for patient treatment, 3D scan data obtained through 3D scanning enables rapid provision of treatment plans and various simulation tasks to patients with high precision.

Meanwhile, a user providing a treatment plan to a patient may simulate a shape before and after orthodontic treatment of the patient based on scan data obtained through 3D scanning. In order to simulate the shape of the patient before and after the orthodontic treatment, a gingiva model among simulation models is generated based on gingiva data among the scan data. By generating the gingiva model, morphing of the gingiva model corresponding to tooth movement occurring in the simulation process may be implemented.

However, when the scan data includes noise data, an inaccurate simulation model may be generated by the noise data or the simulation model may not be generated. The inaccurate simulation model can lead to inaccurate treatment.

In order to prevent generation of the inaccurate simulation model and a failure of simulation model generation, a user may delete the noise data included in the scan data. Conventionally, when the inaccurate simulation model is generated or the simulation model generation fails, the user deletes the noise data and generates the simulation model again. At this time, conventionally, in order to regenerate a simulation model, scan data corrected by deleting noise data is segmented again, and the user has to input new orthodontic plan information. In addition, the process of segmenting the scan data and inputting orthodontic plan information takes a lot of time and system resources, causing inconvenience to users.

### SUMMARY

To solve the above problems, the present disclosure provides a data processing method for generating a simulation model without going through a repetitive segmentation process and an orthodontic plan information input process when scan data is edited to remove noise data.

The technical problems of the present disclosure are not limited to the technical problems mentioned above, and other technical problems not mentioned will be clearly understood by those skilled in the art from the description below.

A data processing method according to the present disclosure may include determining whether a simulation model is to be generated based on scan data of an object obtained by scanning the object and at least one simulation condition for the scan data: when it has been determined that the simulation model is not generated, editing at least a portion of the scan data: and generating the simulation model based on the edited scan data and the at least one simulation condition previously applied in the determining whether the simulation model is to be generated

In addition, the data processing method according to the present disclosure may further include other additional steps including the above-described steps, so that the user can quickly obtain an accurate simulation model.

According to a data processing method according to the present disclosure, even if a simulation model generation fails, a user can quickly acquire a simulation model from edited scan data from which noise data is deleted without performing a repetitive segmentation process and an orthodontic plan information configuration process.

In addition, when scan data is edited, by displaying a portion of the scan data that failed to generate a simulation model, a user can easily remove noise data from the scan data, and quickly acquire the simulation model from the edited scan data from which the noise data is removed.

In addition, in a case in which an area to be deleted is designated to remove noise data, when the area to be deleted includes at least a portion of a tooth area, the area to be deleted may be canceled to prevent the tooth area important for generating a simulation model from being deleted.

### BRIEF DESCRIPTION OF DRAWINGS

FIG. 1 is a schematic configuration diagram illustrating a data processing apparatus in which a data processing method according to the present disclosure is performed.

FIG. 2 is a flowchart illustrating a data processing method according to the present disclosure.

FIG. 3 illustrates an exemplary scan data that is a basis for generating a simulation model in a data processing method according to the present disclosure.

FIG. 4 illustrates a process of aligning scan data in a data processing method according to the present disclosure.

FIG. 5 illustrates a process of configuring orthodontic plan information using segmented scan data in a data processing method according to the present disclosure.

FIG. 6 illustrates a process of generating a portion of a tooth model of a simulation model according to simulation conditions.

FIG. 7 illustrates an exemplary simulation model generated according to simulation conditions.

FIG. 8 illustrates an example of an inaccurate simulation model generated by noise data.

FIG. 9 illustrates a simulation model generation failure message that appears when generation of a simulation model fails.

FIG. 10 is a detailed flowchart illustrating scan data edit step S150 of a data processing method according to the present disclosure.

FIGS. 11 and 12 illustrate a process in which a position of noise data is displayed in scan data edit step S150.

FIG. 13 illustrates a process of removing noise data using a scan data editing tool.

FIG. 14 is a detailed flowchart illustrating noise removal step S153 of a data processing method according to the present disclosure.

FIG. 15 illustrates a state in which an area to be deleted, designated to remove noise data, invades a tooth area of scan data.

FIG. 16 illustrates a state in which a portion of a tooth area is deleted together with noise data in a process of editing scan data.

FIG. 17 illustrates a state in which an area to be deleted, designated to remove noise data, invades a tooth area of scan data in a process of editing scan data in which orthodontic plan information is configured.

FIG. 18 illustrates step S1532a of canceling designation of an area to be deleted.

FIG. 19 is a detailed flowchart illustrating noise removal step S153 of a data processing method according to another embodiment of the present disclosure.

### DETAILED DESCRIPTION

Hereinafter, some embodiments of the present disclosure will be described in detail through exemplary drawings. In adding reference numerals to the components of each drawing, it should be noted that the same reference numerals are assigned to the same components as much as possible even though they are shown in different drawings. In addition, in describing the present disclosure, when it is determined that the detailed description of the related well-known configuration or function may obscure the gist of the present disclosure, the detailed description thereof will be omitted.

The terms, such as first, second, A, B, (a), (b) or the like may be used herein when describing components of the present disclosure. The terms are provided only to distinguish the components from other components, and the essences, sequences, orders, and the like of the components are not limited by the terms. In addition, unless defined otherwise, all terms used herein, including technical or scientific terms, have the same meanings as those generally understood by those skilled in the art to which the present disclosure pertains. The terms defined in the generally used dictionaries should be construed as having the meanings that coincide with the meanings of the contexts of the related technologies, and should not be construed as ideal or excessively formal meanings unless clearly defined in the specification of the present disclosure.

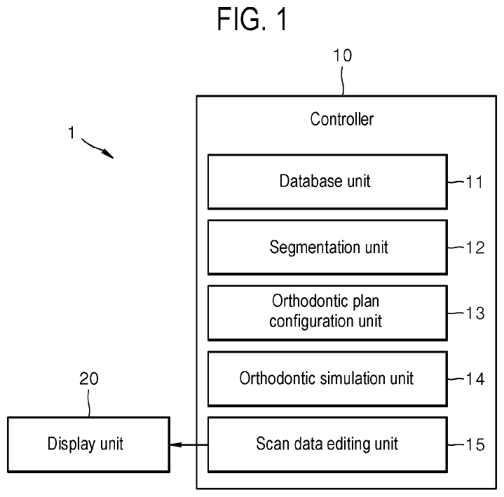

FIG. 1 is a schematic configuration diagram illustrating a data processing apparatus 1 in which a data processing method according to the present disclosure is performed.

Referring to FIG. 1, a data processing apparatus 1 in which a data processing method according to the present disclosure is performed may include a controller 10 and a display unit 20. The controller 10 may be a computing device including a microprocessor capable of processing data steps. For example, the controller 10 may be any one of a PC, a tablet, and a computing device such as a server (or cloud server).

The controller 10 may include a database unit 11. The database unit 11 may store a variety of data including scan data 100. For example, the database unit 11 may store scan data acquired from a scan unit (not shown). At this time, the scan unit may acquire the scan data 100, which is basic data for acquiring a simulation model 200, by scanning an object. For example, the object may include an actual interoral cavity of a patient. However, the object is not limited to the patient's actual interoral cavity, and the object may be an oral model (e.g., a plaster model made by pouring plaster into a mold imitating the patient's interoral cavity) expressing a patient's interoral cavity state for dental treatment of the patient. In addition, the database unit 11 may store logic necessary for performing the data processing method according to the present disclosure, such as logic for generating a simulation model, logic for editing scan data, and logic for tooth segmentation.

In addition, the controller 10 may include a segmentation unit 12. The segmentation unit 12 may segment the scan data. For example, the segmentation unit 12 may segment the scan data into upper jaw data and lower jaw data. In addition, the segmentation unit 12 may segment the scan data into a tooth area and a gingiva area. In particular, the segmentation unit 12 may segment the tooth area of the scan data into individual tooth data. In order for the segmentation unit 12 to segment the tooth area into individual tooth data, tooth information in the human oral cavity may be used. For example, the tooth information may include at least one of curvature information of a tooth, size information of a tooth, and color information of a tooth. For example, the segmentation unit 12 may segment the tooth area into a plurality of pieces of individual tooth data using at least one of characteristic curves, sizes, and colors of molars, premolars, canines, lateral incisors, and central incisors.

In addition, the controller 10 may include an orthodontic plan configuration unit 13. The orthodontic plan configuration unit 13 may configure simulation conditions for performing a simulation based on the segmented scan data. In this case, the simulation may include an orthodontic simulation, and the simulation conditions may include orthodontic plan information. For example, the orthodontic plan configuration unit 13 may configure an orthodontic plan for a specific tooth according to a user's input. For example, the orthodontic plan configuration unit 13 may configure the extraction of an upper left second molar. As another example, the orthodontic plan configuration unit 13 may configure an inlay of a lower right first molar.

In addition, the controller 10 may include an orthodontic simulation unit 14. The orthodontic simulation unit 14 may generate a simulation model 200 by applying simulation conditions including the segmentation information and the orthodontic plan information to the scan data 100. For example, the orthodontic simulation unit 14 may generate the simulation model 200 after orthodontics, in which orthodontic plan information is applied to the scan data 100. The orthodontic simulation unit 14 may generate the simulation model 200 by filling a blank part of the scan data 100 based on the scan data 100 and applying orthodontic plan information to the scan data 100. In addition, the simulation model 200 generated by the orthodontic simulation unit 14 may include an individual tooth model having a contour and a gingiva model covering a portion of the individual tooth model. When the tooth model moves as the orthodontic plan is applied, the gingiva model may also be formed by being morphed in response to the movement of the tooth model.

In addition, the controller 10 may include a scan data editing unit 15. In the orthodontic simulation unit 14, the simulation model 200 may not be generated due to noise data included in the scan data 100. At this time, the scan data editing unit 15 may edit the scan data to generate a simulation model. For example, the scan data editing unit 15 may edit the scan data by deleting the noise data. When the scan data editing unit 15 edits the scan data, the orthodontic simulation unit 14 may generate a simulation model based on the edited scan data. Meanwhile, when generating the simulation model based on the edited scan data, the simulation model may be generated based on previously applied simulation conditions without segmenting the edited scan data or configuring the orthodontic plan information.

Meanwhile, the data processing apparatus 1 according to the present disclosure may include a display unit 20. The display unit 20 may visually display at least some of processes performed by the controller 10. As the display unit 20, at least one of visual display devices such as a monitor, a tablet, and a touch screen may be used. The display unit 20 may display the scan data and/or the simulation model through a user interface screen to be described later.

Hereinafter, a data processing method according to the present disclosure will be described in detail.

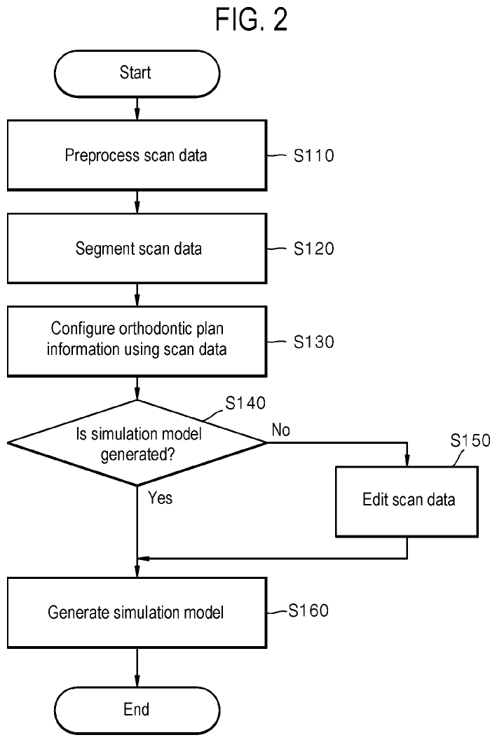

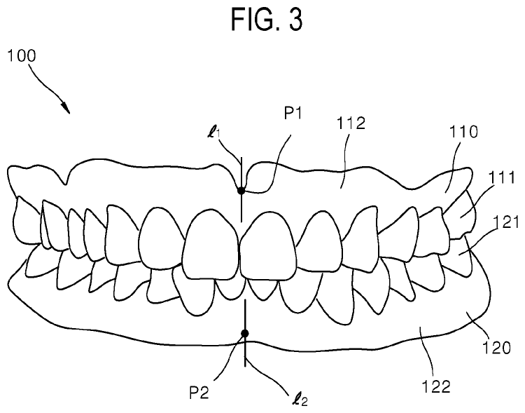

FIG. 2 is a flowchart illustrating a data processing method according to the present disclosure, and FIG. 3 illustrates an exemplary scan data that is a basis for generating a simulation model in a data processing method according to the present disclosure.

Referring to FIGS. 2 and 3, the data processing method according to the present disclosure may include scan data preprocess step S110, segmentation step S120, orthodontic plan configuration step S130, simulation determination step S140, scan data edit step S150, and orthodontic simulation generation step S160.

First, the data processing method according to the present disclosure may include scan data preprocess step S110 of preprocessing acquired scan data 100. The scan data 100 may be 3D data obtained by scanning an object including an interoral cavity having a plurality of teeth and at least one gingiva by the scan unit. The scan unit may be a handheld 3D scanner that the user holds to scan an object at a free scan distance and scan angle with respect to the object. However, the scan unit is not limited to the handheld 3D scanner, and may be a table-type 3D scanner that acquires scan data by placing an object on a tray and rotating and/or tilting the object. The scan data may be previously acquired and stored in a database unit 11 of the controller 10.

In scan data preprocess step S110, the user may edit the scan data 100 before generating a simulation model based on the scan data 100. For example, the user may select and delete at least a portion of the scan data 100 that is visually determined to be noise data. A polygon area selection method may be used as a method of selecting at least a portion of the scan data 100, but is not necessarily limited thereto.

However, scan data preprocess step S110 does not necessarily have to be performed, and the simulation model 200 may be generated directly without editing the acquired scan data 100.

The scan data 100 may represent an object, and may include upper jaw data 110 representing the upper jaw of the object and lower jaw data 120 representing the lower jaw of the object. In addition, the scan data 100 may include tooth areas 111 and 121 representing the teeth of the object and gingiva areas 112 and 122 representing the gingiva of the object.

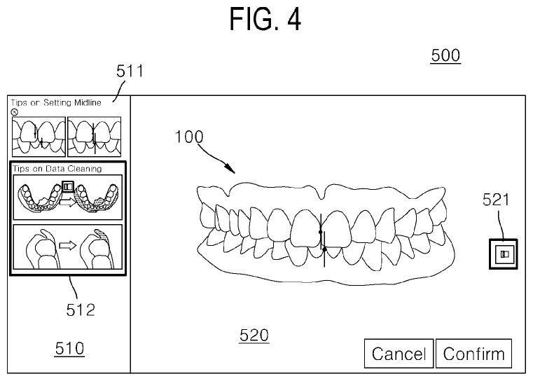

FIG. 4 illustrates a process of aligning scan data in a data processing method according to the present disclosure.

Referring to FIGS. 2 to 4, the scan data 100 may be aligned prior to dividing the tooth areas 111 and 121 and the gingiva areas 112 and 122 of the scan data 100 and segmenting the tooth areas 111 and 121 into a plurality of pieces of individual tooth data. For example, the upper jaw data 110 and the lower jaw data 120 of the scan data 100 may be aligned to have an occlusion shape. In order to align the upper jaw data 110 and the lower jaw data 120, a first alignment point P1 may be designated on the upper jaw data 110, and a second alignment point P2 may be designated on the lower jaw data 120. For example, the first alignment point P1 may be designated between both anterior teeth of the tooth area 111 of the upper jaw data 110, and the second alignment point P2 may be designated between both anterior teeth of the tooth area 121 of the lower jaw data 120. The positions of the upper jaw data 110 and the lower jaw data 120 may be mutually aligned so that a first alignment line 11 extending vertically from the first alignment point P1 and a second alignment line 12 extending vertically from the second alignment point P2 are arranged on a straight line. As the upper jaw data 110 and the lower jaw data 120 of the scan data 100 are aligned, the scan data 100 may be accurately segmented.

Meanwhile, the above-described alignment process may be performed on a user interface screen displayed by the display unit 20. Referring to FIG. 4, a scan user interface screen 500 on which the scan data is displayed from the user interface screen. The scan user interface screen 500 may include a guide unit 510 for displaying guidance for generating the accurate simulation model 200 from the scan data 100 to the user, and a workspace 520 in which the scan data 100 is displayed. The guide unit 510 may display a first guide 511 for aligning the scan data 100 and a second guide 512 for removing noise data from the scan data 100. In addition, the workspace 520 may include a scan data editing tool 521 capable of editing the scan data 521. The user may edit the scan data 100 by selecting the scan data editing tool 521.

When the scan data 100 is aligned, segmentation step S120 may be performed by the segmentation unit 12 of the controller 10. Segmentation step S120 may be performed before simulation determination step S140. In segmentation step S120, the scan data 100 may be segmented into the tooth areas 111 and 121 and the gingiva areas 112 and 122. In addition, in segmentation step S120, segmentation information may be generated by segmenting the tooth areas 111 and 121 of the scan data 100 into a plurality of pieces of individual tooth data. For example, in segmentation step S120, the tooth areas 111 and 121 may be segmented into molar data, premolar data, canine data, lateral incisor data, and central incisor data. In segmentation step S120, in order to segment the tooth areas 111 and 121 into the plurality of pieces of individual tooth data, tooth characteristics of each tooth may be used. For example, the tooth characteristics may include at least one of the curvature of each tooth, the size of each tooth, and the color of each tooth.

Meanwhile, a dental formula (tooth number) may be assigned to the plurality of pieces of segmented individual tooth data according to positions where the individual tooth is disposed. In order to assign the dental formula to the plurality of pieces of individual tooth data, any one of known dental formula assigning methods may be used. For example, the dental formula may be assigned to the plurality of pieces of individual tooth data by any one of known dental formula assigning methods including an FDI method, a universal numbering system method, and a palmer method. Since the scan data 100 is segmented through segmentation step S120, it is possible to configure an orthodontic plan for individual teeth, and to generate a simulation model to which the orthodontic plan is applied.

Hereinafter, orthodontic plan configuration step S130 will be described.

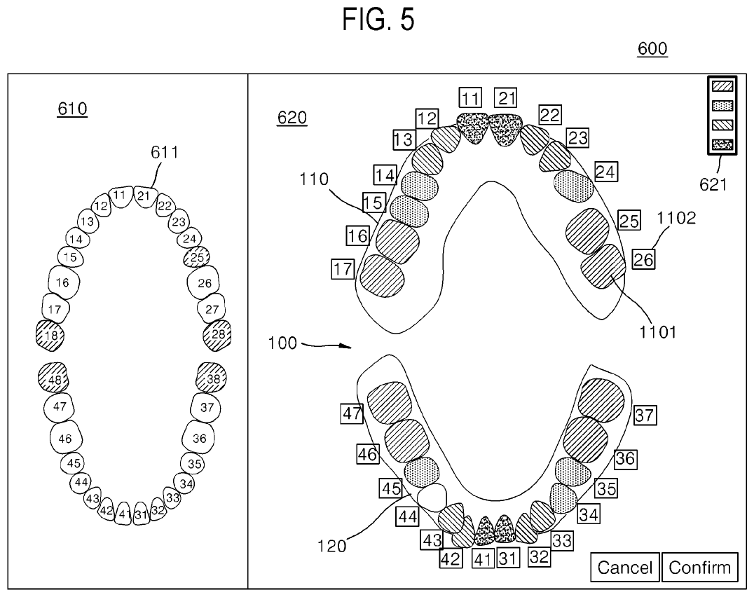

FIG. 5 illustrates a process of configuring orthodontic plan information using segmented scan data 100 in a data processing method according to the present disclosure.

Referring to FIGS. 2 and 5, after segmentation step S120 is performed, orthodontic plan configuration step S130 may be performed by the orthodontic plan configuration unit 13 of the controller 10. In orthodontic plan configuration step S130, orthodontic plan information may be configured using the scan data segmented in segmentation step S120. The orthodontic plan information may include an orthodontic target tooth and a tooth treatment method.

As illustrated in FIG. 5, an orthodontic plan information configuration interface 600 for configuring orthodontic plan information is displayed. The orthodontic plan information configuration interface 600 may include an orthodontic target tooth selection unit 610 and a scan data display unit 620. In addition, in the orthodontic target tooth selection unit 610, the user may select an arranged orthodontic target tooth 611 and may configure an orthodontic plan for the selected orthodontic target tooth 611. In this case, the orthodontic plan may include at least one tooth treatment method such as tooth extraction, tooth removal, inlay, onlay, or prosthesis installation.

In the scan data display unit 620, the scan data 100 may be divided into the upper jaw data 110 and the lower jaw data 120 and displayed, and the scan data 100 may include individual tooth data 1101 and a tooth number 1102 assigned to the individual tooth data 1101. The individual tooth data 1101 may be displayed in a different form according to the type of the segmented tooth. For example, molars may be displayed in a first color (and/or first pattern), premolars may be displayed in a second color (and/or second pattern), canines may be displayed in a third color (and/or third pattern), lateral incisors may be displayed in a fourth color (and/or fourth pattern), and central incisors may be displayed in a fifth color (and/or fifth pattern). The colors and/or patterns for dividing and displaying the individual tooth data 1101 segmented in the scan data display unit 620 may be separately displayed in the form of a legend 621 on one side of the scan data display unit 620.

The user may easily identify the individual tooth data 1101 segmented in the scan data display unit 620, and may configure the orthodontic plan information in the orthodontic target tooth selection unit 610 with reference to the scan data 100 displayed in the scan data display unit 620.

In simulation determination step S140 to be described later, the above-described segmentation information and orthodontic plan information may be used as simulation conditions for generating the simulation model 200 together with the scan data 100. That is, the segmentation information and orthodontic plan information applied to determine whether the simulation model 200 can be generated in simulation determination step S140 may be equally applied in orthodontic simulation generation step S160 performed after editing the noise data by being subjected to scan data edit step S150. Accordingly, the user's convenience can be improved by using the segmentation information and orthodontic plan information applied in simulation determination step S140 as they are without reconfiguring the segmentation information of the scan data and the orthodontic plan information after editing the scan data 100, and the time required for the segmentation process of the scan data and the application process of the orthodontic plan information can be reduced.

Hereinafter, simulation determination step S140 will be described.

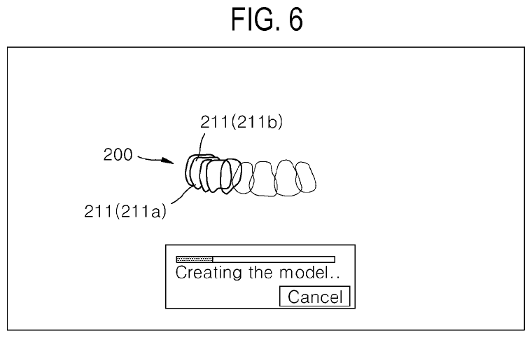

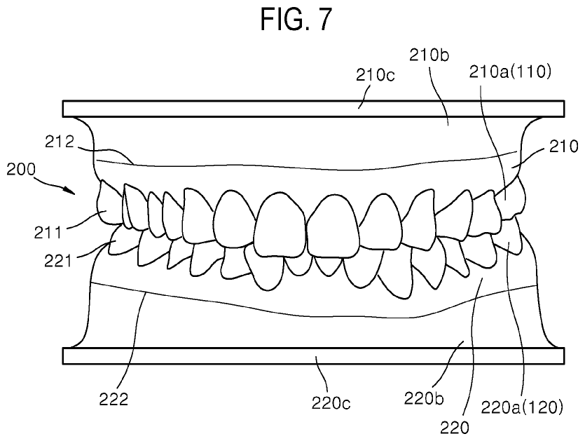

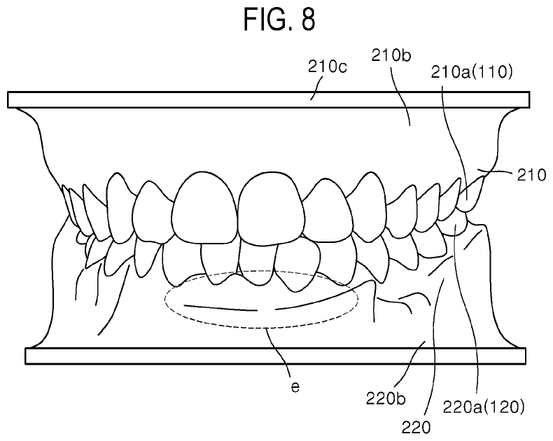

FIG. 6 illustrates a process of generating portions of tooth model 211 or 221 of the simulation model 200 according to simulation conditions, FIG. 7 illustrates an exemplary simulation model 200 generated according to simulation conditions, and FIG. 8 illustrates an example of the inaccurate simulation model 200 generated by noise data.

Referring to FIG. 2 and FIGS. 6 to 8, a data processing method according to the present disclosure may include simulation determination step S140 performed in the orthodontic simulation unit 14 of the controller 10. In simulation determination step S140, whether the simulation model 200 is generated may be determined based on the scan data 100 of an object obtained by scanning the object and at least one simulation condition configured in segmentation step S120 and orthodontic plan configuration step S130 for the scan data 100. For example, simulation determination step S140 may mean determining whether the simulation model 200 is generated by applying simulation conditions to the scan data 100. The simulation model 200 may include an upper jaw model 210 and a lower jaw model 220.

In a case in which noise data is included in the scan data 100, when the simulation model 200 is generated by applying the simulation conditions to the scan data 100, data collision of the simulation model 200 may occur so that the generation of the simulation model 200 may fail. For example, the scan data 100 may include other objects (e.g., a user's finger or a patient's tongue, saliva, soft tissue, etc.) other than the teeth or gingiva of the patient, and the scanned shapes of the other objects may be determined as unnecessary noise data for generating the simulation model 200. Therefore, when the other objects are included in the scan data 100 even if the teeth and gingiva of the subject are scanned satisfactorily, the generation of the simulation model 200 may fail. In addition, when the object is actually inside the oral cavity, since one end of the scan unit (e.g., the tip of the handheld scanner) is difficult to be easily inserted into the posterior teeth of the patient, the difficulty of scanning the posterior teeth of the subject may be high. Accordingly, portions of the gingiva areas 112 and 122 of the scan data 100 representing the posterior teeth of the patient may include noise data generated by an error in the scanning process, and the generation of the simulation model 200 may fail. That is, in simulation determination step S140, it may be determined whether the simulation model 200 can be generated based on the noise data of the gingiva areas 112 and 122.

When the noise data is not included in the scan data 100, or when the noise data does not have a fatal effect on the generation of the simulation module 200 even if the noise data is included in the scan data 100, the orthodontic simulation unit 14 of the controller 10 may generate the simulation model 200 (orthodontic simulation generation step S160). As illustrated in FIGS. 6 and 7, the simulation model 200 may first generate the tooth model 211. The tooth model 211 may be individually generated to correspond to each piece of individual tooth data by the segmented scan data 100. At this time, the tooth areas 111 and 121 of the scan data 100 do not display the entire tooth, and the root of each tooth may be not visible by being hidden by the gingiva areas 112 and 122. Therefore, in the orthodontic simulation step S160, the tooth areas 111 and 121 of the scan data 100 may be supplemented using information on the individual tooth data. For example, the tooth model 211 may include a scan data-based tooth model 211a generated by converting the tooth areas 111 and 121 of the scan data 100 and a virtual data-based tooth model 211b that is integrally formed with the scan data-based tooth model 211a and is accommodated in gingiva models 212 and 222. The scan data-based tooth model 211a and the virtual data-based tooth model 211b may be integrally combined to form an individual closed tooth shape.

In addition, in orthodontic simulation step S160, the gingiva regions 112 and 122 of the scan data 100 may be supplemented. For example, the simulation model 200 may include scan data-based simulation models 210a and 220a including portions of the tooth models 211 and 221 and the gingiva models 212 and 222, and virtual data-based simulation models 210b and 220b filled from the scan data-based simulation models 210a and 220a to an upper jaw model boundary 210c and a lower jaw model boundary 220c, respectively. At this time, the virtual data-based simulation models 210b and 220b may be generated according to predetermined gingiva model supplementation logic to generate the gingiva models 212 and 222 together with the gingiva areas 112 and 122 of the scan data 100.

However, referring to FIG. 8, in the lower jaw model 220 of the simulation model 200, noise data may be included in the gingiva area 122 of the lower jaw data 120, so that an unnatural model error area e may be generated. The model error area e may express a different shape from the patient's actual interoral cavity, so that there is a concern that inaccurate simulation results may be provided to the patient. Accordingly, in simulation determination step S140, it may be determined that the generation of the simulation model 200 has failed when the model error area e is generated or a collision occurs within the simulation model 200.

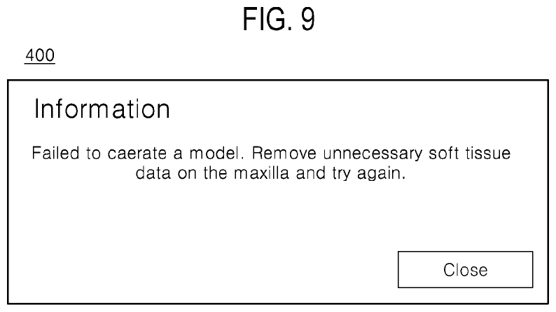

FIG. 9 illustrates a simulation model generation failure message 400 that appears when generation of the simulation model 200 fails.

Referring to FIG. 9, when the generation of the simulation model 200 fails, in simulation determination step S140, the simulation model generation failure message 400 may be output through the display unit 20. The user can easily recognize that noise data is included in the scan data 100 by identifying the simulation model generation failure message 400.

Hereinafter, scan data edit step S150 will be described in detail.

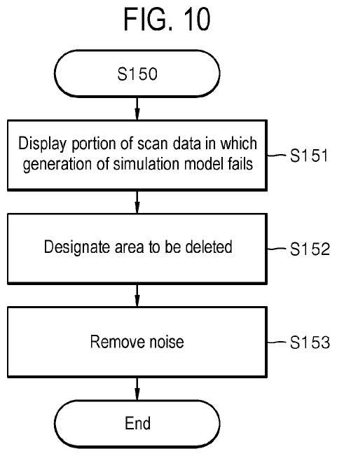

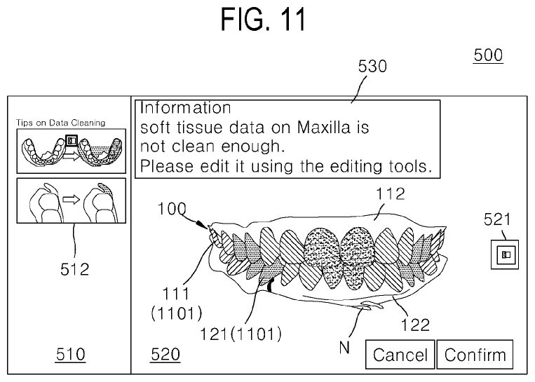

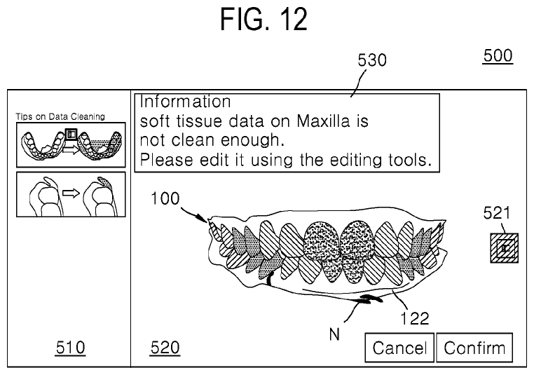

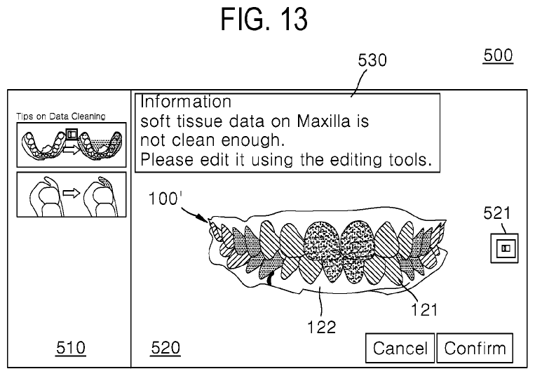

FIG. 10 is a detailed flowchart illustrating scan data edit step S150 of a data processing method according to the present disclosure, and FIGS. 11 and 12 illustrate a process in which a position of noise data N is displayed in scan data edit step S150. In addition, FIG. 13 illustrates a process of removing noise data N using the scan data editing tool 521.

Referring to FIG. 2 and FIGS. 10 to 12, when it is determined that the generation of the simulation model 200 has failed in simulation determination step S140, scan data edit step S150 may be performed by the scan data editing unit 15 of the controller 10. For example, in scan data edit step S150, at least a portion of the scan data 100 may be edited when the generation of the simulation model 200 fails.

Scan data edit step S150 may include model generation failure reason display step S151 of displaying a portion of the scan data that causes a failure in the generation of the simulation model 200. In model generation failure reason display step S151, the type of the scan data 100 for which the generation of the simulation model 200 fails in simulation determination step S140 may be displayed. For example, as illustrated in FIG. 11, the scan user interface screen 500 may further include a guidance window 530. The guidance window 530 may display the type of the scan data 100 for which the generation of the simulation model 200 has failed. For example, the guidance window 530 may display a portion where the generation of the simulation model 200 has failed in simulation determination step S140. More specifically, the guidance window 530 may display whether the type of the scan data 100 for which the generation of the simulation model 200 has failed is the upper jaw data 110 or the lower jaw data 120. In this manner, since the type of the scan data 100 for which the generation of the simulation model 200 has failed is displayed through the guidance window 530, the user can easily edit the scan data 100 and may quickly acquire the normal simulation model 200.

In addition, referring to FIGS. 10 and 12, the type of the scan data 100 indicated as a failure in the generating the simulation model 200 in simulation determination step S140 may include noise data N. More specifically, the guidance window 530 may display the type (upper jaw data or lower jaw data) of the scan data 100 including the noise data N, and the workspace 520 may directly display the shape of the noise data N included in the scan data 100. When the user selects the scan data editing tool 521 to enter the process of editing the scan data 100, the noise data N may be expressed as at least one of a predetermined color, a predetermined pattern, and a predetermined mark in scan data edit step S150. For example, the noise data N may be expressed as a fluorescent color. In addition, a position where the noise data N exists may be indicated by an arrow (not shown). Accordingly, the user can easily grasp the position where the noise data N exists in the scan data 100 displayed on the scan user interface screen 500.

Referring to FIG. 13, the user may remove the noise data by selecting the scan data editing tool 521. Accordingly, edited scan data 100′ from which the noise data is removed may be generated. Meanwhile, the process of removing the noise data may be automatically performed, but is not necessarily limited thereto, and a corresponding portion may be removed by designating an area to be deleted, which will be described later.

For example, the scan data edit step S150 may include deletion target area designation step S152. Deletion target area designation step S152 may mean designating an area A to be deleted, which is at least a portion of the scan data 100, so that at least a portion of the noise data N is included. The area A to be deleted may be a polygonal area generated by designating vertices of a polygon by the user. Alternatively, the area A to be deleted may be a circular area or an elliptical area.

When the area A to be deleted is designated, noise removal step S153 may be performed. In noise removal step S153, the area A to be deleted may be deleted from the scan data 100. By deleting the area A to be deleted from the scan data 100, a cause of a failure in generation of the edited scan data 100′ into the simulation model 200 may be eliminated.

As described above, after the scan data edit step S150 is performed, orthodontic simulation generation step S160 may be performed. Orthodontic simulation generation step S160 may be performed in the orthodontic simulation unit 14, and the simulation model 200 may be generated based on the edited scan data 100′ and the simulation conditions previously applied in simulation determination step S140. The noise data N may be generally included in the gingiva areas 112 and 122, and the presence and removal of the noise data N does not affect segmentation step S120 in which the tooth areas 111 and 121 are segmented into individual pieces of tooth data and orthodontic plan configuration step S130 in which the orthodontic plan is configured using the individual pieces of tooth data. Therefore, when the orthodontic simulation generation step S160 is performed based on the edited scan data 100′, the segmentation information and orthodontic plan information of the scan data 100 before editing may be applied as they are. That is, when the edited scan data 100′ is generated by editing the noise data N included in the scan data 100 and orthodontic simulation generation step S160 is performed, the segmentation process may be prevented from being performed again on the edited scan data 100′. When the segmentation process is performed again on the edited scan data 100′, the segmentation information generated according to the segmentation process of the scan data 100 and the segmentation information generated according to the segmentation process of the edited scan data 100′ may be different from each other. For example, when the segmentation process is performed again on the edited scan data 100′, a dental formula given according to the segmentation process of the edited scan data 100′ may be different from a dental formula given according to the segmentation process of the scan data 100, and new orthodontic plan information corresponding to the changed dental formula of the edited scan data 100′ should be configured. In the present disclosure, segmentation step S120 for the edited scan data 100′ may not be performed again, and the segmentation information and orthodontic plan information for the scan data 100 before editing may be preserved and applied equally to the edited scan data 100′, whereby system resources used in segmentation step S120 and orthodontic plan configuration step S130 may be saved and the time required to generate the simulation model 200 may be reduced.

Hereinafter, noise removal step S153 will be described in detail.

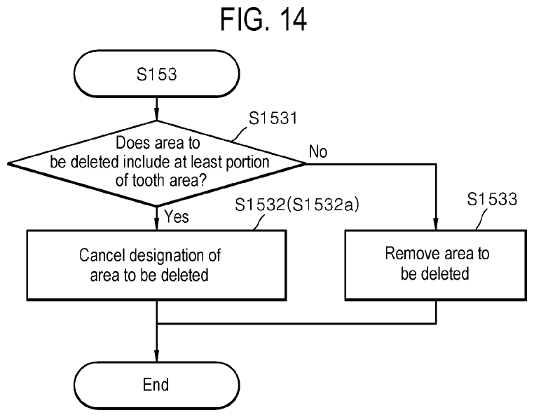

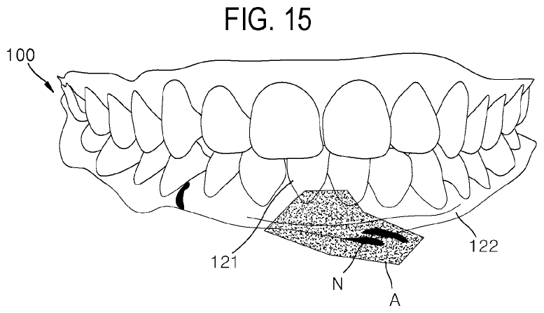

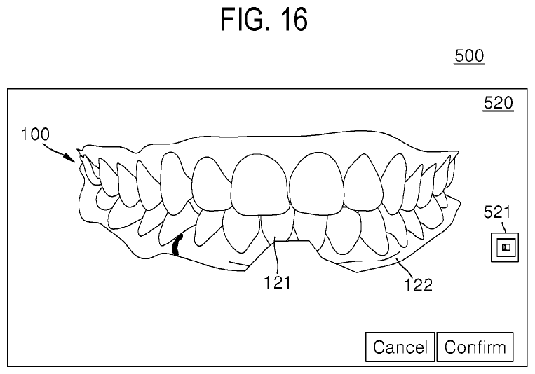

FIG. 14 is a detailed flowchart illustrating noise removal step S153 of a data processing method according to the present disclosure, FIG. 15 illustrates a state in which an area A to be deleted, designated to remove noise data N, invades the tooth areas 111 and 121 of the scan data 100, and FIG. 16 illustrates a state in which portions of the tooth areas 111 and 121 are deleted together with noise data N in a process of editing the scan data 100.

Referring to FIGS. 14 to 16, in the process of editing the scan data 100, the area A to be deleted may include at least portions of the tooth areas 111 and 121. At this time, when the area A to be deleted is removed, the portions of the tooth areas 111 and 121 may be also removed, and thus areas necessary for the simulation model 200 may be removed by mistake. When the area A to be deleted includes not only the noise data N but also a portion of the tooth area 121 as shown in FIG. 15, a portion of the tooth area 121 may be removed as shown in FIG. 16. In this manner, when the portions of the tooth areas 111 and 121 are removed, the possibility of generating the inaccurate simulation model 200 may increase.

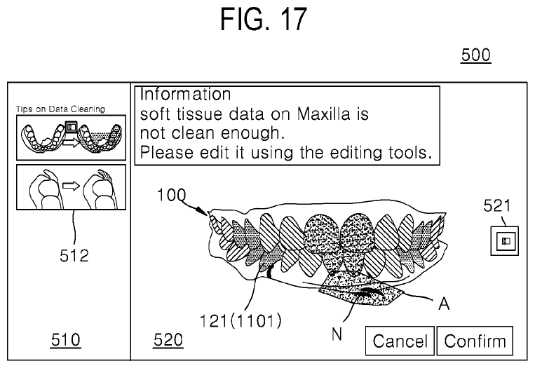

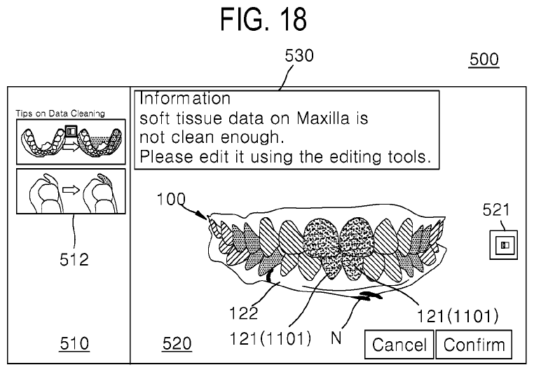

FIG. 17 illustrates a state in which an area A to be deleted, designated to remove noise data N, invades the tooth areas 111 and 121 of scan data in a process of editing the scan data 100 in which an orthodontic plan is configured, and FIG. 18 illustrates step S1532a of canceling designation of an area to be deleted.

In order to solve the above problem, noise removal step S153 may include deletion target area determination step S1531 of determining whether the area to be deleted includes at least a portion of the tooth area. Referring to FIGS. 14 and 17, in the above-described segmentation step S120, the tooth areas 111 and 121 of the scan data 100 may be distinguished from the gingiva areas 112 and 122, and the tooth areas 111 and 121 may be segmented into individual piece of tooth data 1101 again. Meanwhile, the segmentation information generated in segmentation step S120 and the orthodontic plan information generated in orthodontic plan configuration step S130 may be applied to the scan data 100 in noise removal step S153. Accordingly, in deletion target area determination step S1531 performed to edit the scan data 100 after it is determined that the generation of the simulation model 200 has failed, it may be determined whether the designated area A to be deleted includes at least portions of the tooth areas 111 and 121 of the scan data 100 or the individual piece of tooth data 1101.

At this time, when the area A to be deleted includes the at least portions of the tooth areas 111 and 121, steps S1532 and S1532a of canceling designation of the area to be deleted may be performed. For example, when the user inputs a command to remove the area A to be deleted including the at least portions of the tooth areas 111 and 121, the designation of the designated area A to be deleted may be canceled, and the area A to be deleted may not be removed from the scan data 100. The user may remove the noise data N by redesignating the area A to be deleted so that the tooth areas 111 and 121 are not included. When the area A to be deleted does not include the tooth areas 111 and 121, step S1533 of removing the area to be deleted may be performed so that the scan data 100 can be edited.

Accordingly, by preventing the removal of the tooth areas 111 and 121 necessary for generating the simulation model 200, the user may designate that the area A to be deleted includes only the noise data N and portions of the gingiva areas 112 and 122, and may acquire the accurate simulation model 200.

Hereinafter, a data processing method according to another embodiment of the present disclosure will be described.

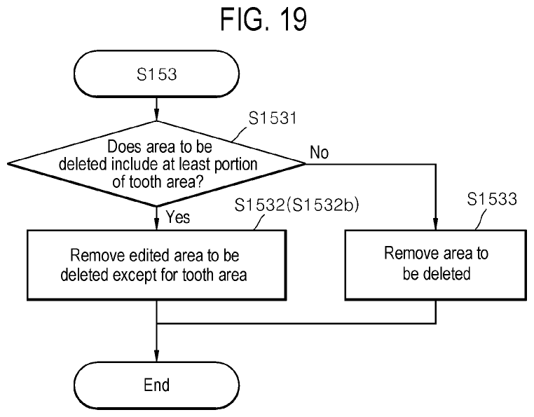

FIG. 19 is a detailed flowchart illustrating noise removal step S153 of a data processing method according to another embodiment of the present disclosure.

Referring to FIG. 19, the data processing method according to another embodiment of the present disclosure may differ from the above-described data processing method in some of the detailed steps of noise removal step S153. Noise removal step S153 may include step S1531 of determining whether the area to be deleted includes at least a portion of the tooth area, and a process of determining the area A to be deleted is the same as described above.

However, in the data processing method according to another embodiment of the present disclosure, when it is determined that the area A to be deleted includes at least portions of the tooth areas 111 and 121, steps S1532 and S1532b of removing a portion of the area to be deleted may be performed. For example, in step S1532b of removing a portion of the area to be deleted, an area except for the tooth areas 111 and 121 of the area A to be deleted may be removed. More specifically, in step S1532b of removing a portion of the area to be deleted, areas except for the tooth areas 111 and 121 of the area A to be deleted and a tooth adjacent area formed within a predetermined distance from the tooth areas 111 and 121 may be removed from the scan data 100. In this case, the tooth adjacent area may refer to a partial area of the gingiva areas 112 or 122 included within a predetermined distance from the contours of the tooth areas 111 or 121. In this manner, by removing the area to be deleted that is modified to exclude the tooth areas 111 and 121 necessary for generating the simulation model 200 and the tooth adjacent area, noise data N may be stably removed, and the accurate simulation model 200 may be generated to provide optimal treatment to a patient.

The above description is merely illustrative of the technical idea of the present disclosure, and those of ordinary skill in the art to which the present disclosure pertains will be able to make various modifications and variations without departing from the essential characteristics of the present disclosure.

Therefore, the embodiments disclosed in the present disclosure are not intended to limit the technical idea of the present disclosure, but to explain, and the scope of the technical idea of the present disclosure is not limited by these embodiments. The scope of protection of the present disclosure should be interpreted by the claims below, and all technical ideas within the scope equivalent thereto should be construed as being included in the scope of the present disclosure.

1. A data processing method comprising:
determining whether a simulation model is to be gene rated based on scan data of an object obtained by scanning the object and at least one simulation condition for the scan data;
when it has been determined that the simulation model is not generated, editing at least a portion of the scan data; and
generating the simulation model based on the edited scan data and the at least one simulation condition previously applied in the determining whether the simulation model is to be generated.

2. The data processing method of claim 1, wherein the object comprises an interoral cavity having a plurality of teeth and at least one gingiva, the scan data comprises a tooth area representing a plurality of teeth and a gingiva area representing the at least one gingiva, and the gingiva area comprises noise data.

3. The data processing method of claim 2, wherein the determining whether the simulation model is to be generated comprises determining whether the generation of the simulation model fails based on the noise data of the gingiva area.

4. The data processing method of claim 2, further comprising generating segmentation information by segmenting the tooth area of the scan data into a plurality of pieces of individual tooth data before the determining whether the simulation model is to be generated,
wherein the at least one simulation condition comprises the segmentation information.

5. The data processing method of claim 4, further comprising configuring orthodontic plan information using the segmented scan data after the generating the segmentation information,
wherein the at least one simulation condition further comprises the orthodontic plan information.

6. The data processing method of claim 2, wherein the editing at least a portion of the scan data comprises displaying a type of the scan data for which the generation of the simulation model has failed.

7. The data processing method of claim 6, wherein the type of the scan data displayed as a failure in the generation of the simulation model comprises upper jaw data and lower jaw data.

8. The data processing method of claim 6, wherein the type of the scan data displayed as a failure in the generation of the simulation model comprises the noise data, and the noise data is expressed as at least one of a predetermined color, a predetermined pattern, and a predetermined mark.

9. The data processing method of claim 2, wherein the editing at least a portion of the scan data comprises:
designating an area to be deleted that is at least a portion of the scan data so that at least a portion of the noise data is included; and
removing the area to be deleted from the scan data.

10. The data processing method of claim 9, wherein, when the area to be deleted comprises at least a portion of the tooth area, the area to be deleted is not removed from the scan data.

11. The data processing method of claim 9, wherein, when the area to be deleted comprises at least a portion of the tooth area, areas except for the tooth area of the area to be deleted and a tooth adjacent area formed within a predetermined distance from the tooth area are removed from the scan data.

12. An apparatus comprising a controller configured to:
determine whether a simulation model is to be generated based on scan data of an object obtained by scanning the object and at least one simulation condition for the scan data;
when it has been determined that the simulation model is not generated, edit at least a portion of the scan data; and
generate the simulation model based on the edited scan data and the at least one simulation condition previously applied in the determining whether the simulation model is to be generated.

13. The apparatus of claim 12, wherein the object comprises an interoral cavity having a plurality of teeth and at least one gingiva, the scan data comprises a tooth area representing a plurality of teeth and a gingiva area representing the at least one gingiva, and the gingiva area comprises noise data.

14. The apparatus of claim 13, wherein the controller is configured to determine whether the generation of the simulation model fails based on the noise data of the gingiva area.

15. The apparatus of claim 13, wherein the controller is further configured to generate segmentation information by segmenting the tooth area of the scan data into a plurality of pieces of individual tooth data before the determining whether the simulation model is to be generated, and
wherein the at least one simulation condition comprises the segmentation information.

16. The apparatus of claim 15, wherein the controller is further configured to configure orthodontic plan information using the segmented scan data after the generating the segmentation information, and
wherein the at least one simulation condition further comprises the orthodontic plan information.

17. The apparatus of claim 13, wherein the controller is configured to display a type of the scan data for which the generation of the simulation model has failed, and
wherein the type of the scan data displayed as a failure in the generation of the simulation model comprises upper jaw data, lower jaw data, and the noise data, and the noise data is expressed as at least one of a predetermined color, a predetermined pattern, and a predetermined mark.

18. The apparatus of claim 13, wherein the controller is configured to:
designate an area to be deleted that is at least a portion of the scan data so that at least a portion of the noise data is included; and
remove the area to be deleted from the scan data.

19. The apparatus of claim 18, wherein, when the area to be deleted comprises at least a portion of the tooth area, the area to be deleted is not removed from the scan data.

20. The apparatus of claim 18, wherein, when the area to be deleted comprises at least a portion of the tooth area, areas except for the tooth area of the area to be deleted and a tooth adjacent area formed within a predetermined distance from the tooth area are removed from the scan data.
